(19) United States
(12) Patent Application Publication
(10) Pub. No.: US 2021/0186614 A1
(43) Pub. Date: Jun. 24, 2021

(54) STABILIZATION OF BONE POSITIONS DURING TOTAL JOINT ARTHROPLASTY

(71) Applicant: THINK SURGICAL, INC., Fremont, CA (US)

(72) Inventors: Micah Forstein, Fremont, CA (US); Steve Henderson, Fremont, CA (US)

(73) Assignee: THINK SURGICAL, INC., Fremont, CA (US)

(21) Appl. No.: 17/057,338

(22) PCT Filed: May 22, 2019
(86) PCT No.: PCT/US2019/033606

§ 371 Date: Nov. 20, 2020

Related U.S. Application Data
(60) Provisional application No. 62/675,478, filed on May 23, 2018.

(51) Int. Cl. A61B 34/10; A61B 90/00; A61B 34/20; A61B 34/30; A61F 2/46; G16H 20/40; G16H 50/50

(52) U.S. Cl. CPC A61B 34/10; A61B 90/06; A61B 34/20; A61B 34/30; A61F 2/461; G16H 20/40; A61B 17/1675; A61B 2034/105; A61B 2090/065; A61B 2562/0261; A61B 2562/0223; A61B 2562/0247; A61B 2034/2055; G16H 50/50

(57) ABSTRACT

A computerized system and method is provided for stabilizing a first bone relative to a second bone during robotic based total joint arthroplasty. A plurality of cut paths are determined, either pre-operatively or intra-operatively using three-dimensional (3-D) virtual bone models, relative to the first bone and/or second bone in order to modify the bone(s) to receive an implant in a desired position and orientation. At least one stability region is identified between the two bones, where one or more cut paths are adjusted to avoid the at least one stability region. The first bone is therefore stabilized against the second bone at the at least one stability region while the remaining cut paths are executed around the stability region. Finally, the at least one stability region is removed once the reaming cut paths are completed and an implant is placed on the modified bone(s).

### RELATED APPLICATIONS

This application claims priority benefit of U.S. Provisional Application Ser. No. 62/675,478 filed 23 May 2018, the contents of which are hereby incorporated by reference.

### TECHNICAL FIELD

The present invention generally relates to computer-assisted orthopedic surgery, and more particularly to the stabilization of two bones in a joint while performing total joint arthroplasty.

### BACKGROUND

Throughout a lifetime, bones and joints become damaged and worn through normal use, disease, and traumatic events. Arthritis is a leading cause of joint damage, which can cause cartilage degradation, pain, swelling, stiffness, and bone loss overtime. If the pain associated with the dysfunctional joint is not alleviated by less-invasive therapies, the joint may need to be replaced with a procedure called total joint arthroplasty (TJR). TJR is an orthopedic surgical procedure in which the typically-worn articular surfaces of the joint are replaced with prosthetic components, or implants. TJR typically requires the removal of the articular cartilage of the joint including a varying amount of bone, with the amount depending on the joint and the implant being used. This cartilage and bone is then replaced with synthetic implants, typically metal and plastic, which form the new synthetic joint surfaces.

The accurate placement and alignment of the implants on the bone is a large factor in determining the success of a TJR procedure. A slight misalignment may result in poor wear characteristics, reduced functionality, poor clinical outcomes, and decreased longevity. Therefore, several devices, systems, and methods have been developed to accurately prepare the bone to receive the implants in a desired position and orientation (POSE). In some methods, a cutting jig or alignment guide may be used to accurately position and orient a cutting tool such as a saw, drill, or reamer to create the bone cuts. In other methods, the cuts may be made with the aid of a computer-assisted surgical device (e.g., tracked surgical instruments, robotics).

In any case, manipulating and positioning the bones to make the cuts may be particularly difficult especially when trying to cut the bone in hard-to-reach surgical areas (e.g., the posterior bone cuts in total knee arthroplasty). One factor contributing to this difficulty is the forces produced by the surrounding tissues and ligaments. With reference to prior art FIG. 1, a knee joint ‘KJ’ is shown prior to undergoing total knee arthroplasty. The knee joint ‘KJ’ includes the femur ‘F’, tibia ‘T’, and several ligaments including the medial collateral ligament ‘MCL’ and the lateral collateral ligament ‘LCL’. In particular, the ligaments and surrounding tissues are naturally pre-loaded to force the bones towards one another (as denoted by the arrows). Once bone in the proximal condyles and or the proximal tibial plateau is removed, the stability of the bones is directly dependent on the presence of an external force to counter the ligament pre-load (e.g., rigid fixation of the bones to a surgical table or surgical robot, a distraction device positioned between the bones, and/or a user manually distracting the bones from one another). Otherwise the bones are forced together, which makes cutting directly between the bones challenging, especially for robotic surgical procedures.

One such robotic surgical system is the TSOLUTION ONE® Surgical System (THINK Surgical, Inc., Fremont, Calif.), which aids in the planning and execution of total hip arthroplasty (THA) and total knee arthroplasty (TKA). The TSOLUTION ONE® Surgical System includes: a pre-operative planning software program to generate a surgical plan using an image data set of the patient's bone and computer-aided design (CAD) files of several implants; and an autonomous surgical robot that precisely mills the bone to receive an implant according to the surgical plan. The robot is equipped with a force sensor to sense the forces exerted on an end-effector of the robot. As a safety precaution, any external forces sensed on the end-effector above a threshold force causes the robot arm to freeze. In TKA, a force-freeze may be triggered when the tibia ‘T’ is forced towards the femur ‘F’ as the end-effector removes bone from the femur ‘F’. These force-freezes delay the surgical procedure, which can increase surgical complications and operating costs. Traditionally, this problem was avoided by fixing the bones directly to the robot by a series of clamps and rods. Recently however, there is a desire to reduce the amount of bone fixation to permit the users to adjust the bone positions on-the-fly. In addition, the rods are screwed into the bone which may weaken the bone. Alternatively, a distraction device may be used to force the bones apart but at the expense of crowding the surgical site and potentially colliding with the end-effector.

Thus, there is a need in the art for a system and method for stabilizing a first bone relative to a second bone in a joint during total joint arthroplasty.

### SUMMARY OF THE INVENTION

A method is provided for stabilizing a first bone relative to a second bone in a joint during total joint arthroplasty. The method includes determining a plurality of cut paths relative to the first bone in order to modify the first bone to receive an implant thereon, identifying one or more stability regions between the first bone and the second bone. Subsequently, one or more cut paths are adjusted to avoid at least one of the one or more stability regions, while the first bone is stabilized against the second bone at the at least one of the one or more stability regions while the remaining cut paths are executed around the at least one of the one or more stability regions. Finally, and the at least one of the one or more stability regions is removed once the remaining cut paths are completed.

A system is provided for stabilizing a first bone relative to a second bone in a joint during total joint arthroplasty. The system includes a pressure-sensing device to aid in identifying the at least one stability region, a manipulator arm supporting an end-effector, and a computing system that includes a plurality of cut files stored therein, each cut file having a set of cut paths to be executed by the manipulator arm. The computing system selects a specific cut file based on the output from the pressure-sensing device, the specific cut file having a set of cut paths that avoid the at least one stability region.

A computer-assisted surgical system is provided for stabilizing a first bone relative to a second bone in a joint during total joint arthroplasty. The system includes a computer-assisted surgical device having an end-effector, a computing system comprising operational data to be executed by the surgical device to remove bone according to a surgical plan, and a wedge to be inserted between two bones to stabilize the bones while the surgical device removes bone.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 depicts a prior art knee joint;

FIG. 2 depicts a flowchart of a method for stabilizing two bones during TJR in accordance with embodiments of the invention;

FIG. 3A depicts a pressure-sensing device in accordance with embodiments of the invention;

FIG. 3B depicts the pressure-sensing device of FIG. 3A between a femur and a tibia in accordance with embodiments of the invention.

FIG. 3C depicts a series of pressure maps produced from a pressure-sensing device in accordance with embodiments of the invention;

FIG. 4A depicts a femur and tibia having a plurality of cut paths and a stability region determined and identified in relation thereto in accordance with embodiments of the invention;

FIG. 4B depicts a femur and tibia stabilized at a stability region in accordance with embodiments of the invention;

FIGS. 5A-5C depict a femur and tibia stabilized with a series of wedges in accordance with embodiments of the invention; and

FIG. 6 depicts a robotic surgical system in accordance with embodiments of the invention.

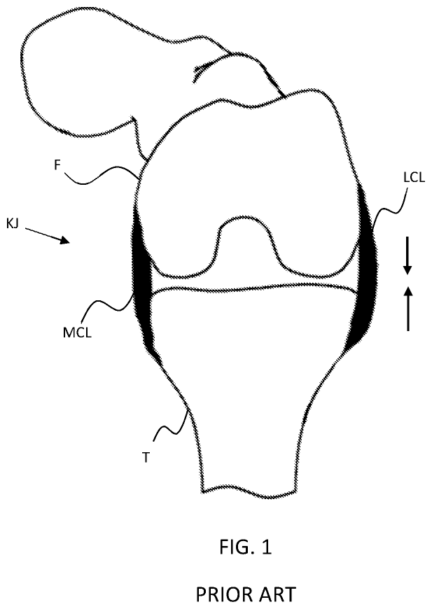

### DETAILED DESCRIPTION

The present invention has utility as a system and method for stabilizing a first bone relative to a second bone during total joint arthroplasty. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Further, it should be appreciated that although the systems and methods described herein make reference to the knee, the systems and methods may be applied to other bones and joints in the body illustratively including the hip, ankle, elbow, wrist, skull, and spine, as well as revision of initial repair or replacement of any of the aforementioned bones or joints.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “registration” refers to the determination of the position and orientation (POSE) and/or coordinate transformation between two or more objects or coordinate systems such as a computer-assist device, a bone, pre-operative bone data, surgical planning data (e.g., an implant model, a computer software “cut-file” to identify a cutting path, virtual boundaries, virtual planes, cutting parameters associated with or defined relative to the pre-operative bone data, etc.), and any external landmarks (e.g., a tracking marker array, an anatomical landmark, etc.) associated with the bone, if such landmarks exist. Various methods of registration are well known in the art and are described in, for example, U.S. Pat. Nos. 6,033,415; 8,010,177; and 8,287,522; which patents are hereby incorporated herein by reference.

As used herein, the term “real-time” refers to the processing of input data within milliseconds such that calculated values are available within 10 seconds of computational initiation.

As used herein, the term “stability region” generally refers to a region of contact between a first bone and a second bone. The region may include direct bone-to-bone contact, as well as indirect contact by intervening structures (e.g., articular cartilage). The stability region is preferably a region of contact between two bones having pressure therebetween that are higher compared to neighboring regions.

Also described herein are ‘computer-assisted surgical devices’. A computer assisted surgical device refers to any device/system requiring a computer to aid in a surgical procedure. Examples of a computer-assisted surgical device include a tracking system, tracked passive instruments, active or semi-active hand-held surgical devices and systems, autonomous/active serial-chain manipulator systems, haptic serial chain manipulator systems, parallel robotic systems, or master-slave robotic systems, as described in U.S. Pat. Nos. 5,086,401, 7,206,626, 8,876,830, 8,961,536, and 9,707,043 and PCT. Publication WO2016049180.

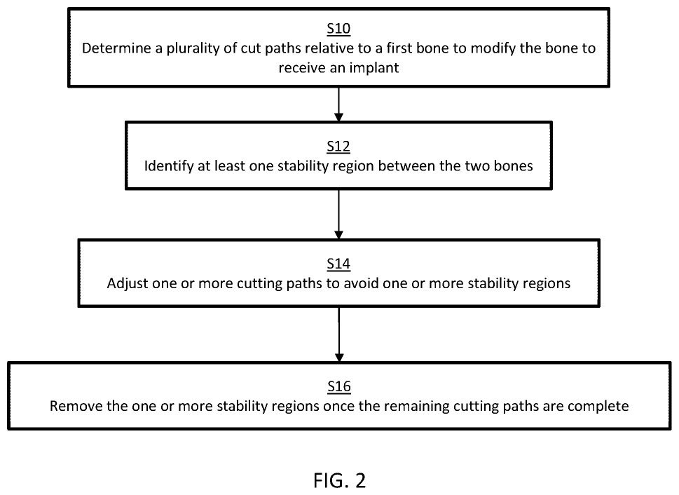

With reference now to the figures, FIG. 2 depicts an embodiment of an inventive method for stabilizing the position of a first bone relative to a second bone during TJR. A plurality of cut paths are determined relative to the first bone and/or second bone in order to modify the bone(s) to receive an implant in a desired POSE (Block S10). At least one stability region is identified between the two bones (Block S12), where one or more of the plurality of cut paths are adjusted to avoid the at least one stability region (Block S14). The first bone is therefore stabilized against the second bone at the at least one stability region while the remaining cut paths are executed around the stability region. Finally, the at least one stability region is removed (Block S16) once the reaming cut paths are completed and an implant may be placed on the modified bone(s). Details of the method are further described below.

The plurality of cut paths may be determined relative to the bone either pre-operatively or intra-operatively (Block S10). In a specific inventive embodiment, the cut paths are determined relative to a bone in a planning software program according to the following. Three-dimensional (3-D) virtual bone models of the first bone and second bone are generated and provided to the user in the planning software. The 3-D models may be generated from an image data set of the first bone and second bone acquired with an imaging modality including, for example, computed tomography (CT), magnetic resonance imaging (MRI), X-Ray, or ultrasound. Alternatively, the 3-D models may be generated by collecting a plurality of points directly on the bone intra-operatively, which is typical of many imageless computer-assisted surgical procedures. Computer-aided design (CAD) files of the implants (implant models) are also provided to the user in the planning software. The user may manipulate the implant models relative to the bone models to designate the best fit, fill, and/or position for the implants on the bones. Subsequently, based on the designation, the cut paths are determined relative to the bone. In one example, the cut paths are determined based on the intersection of the implant model with the bone models (e.g., the position of a planar cut path is determined based on the position of the planar surfaces of a femoral knee component intersecting with the bone model). In another example, the cut paths are determined and pre-defined based on the geometry of an implant and therefore the positions of the cut paths are automatically determined once the user designates the desired POSE for the implant model(s) relative to the bone model(s). In a further example, the cut paths are determined relative to the bone using conventional non-computer assisted planning techniques with two-dimensional (2-D) x-rays. In an even further example, the cut paths are determined relative to the bone intra-operatively using cutting jigs, slots, or other fixtures.

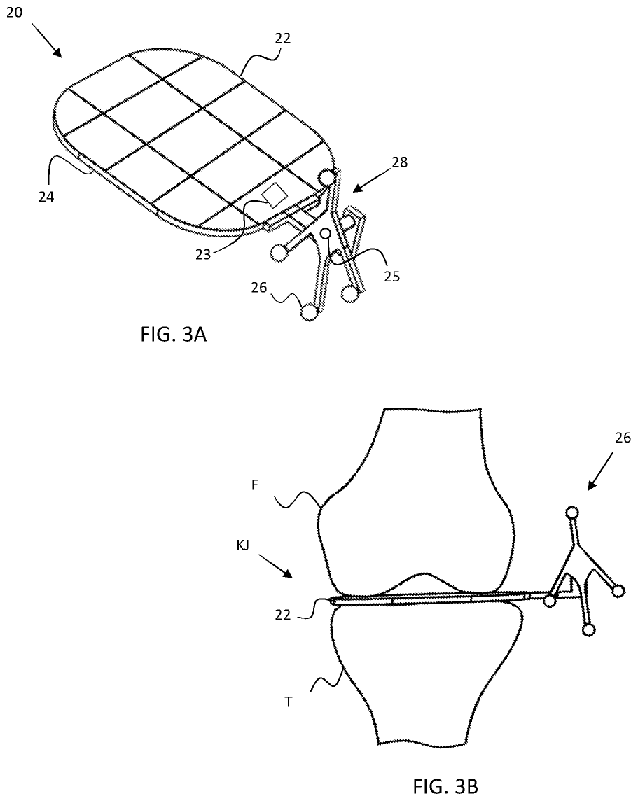

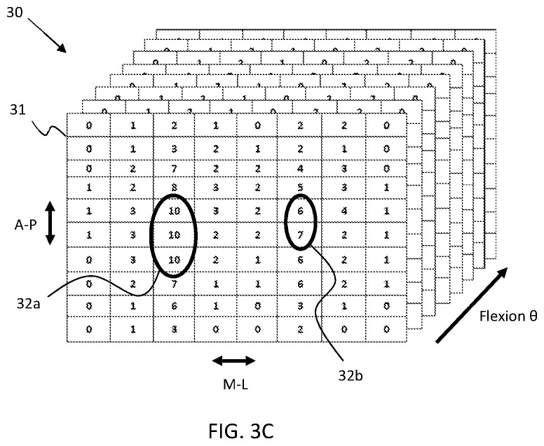

The at least one stability region may be identified (Block S12) using several different methods. In a particular inventive embodiment, with reference to FIGS. 3A-3C, a pressure-sensing device 20 is shown to aid in identifying at least one stability region. The pressure-sensing device 20 is configured to determine the pressures at various regions between two bones. In a particular inventive embodiment, the pressure-sensing device 20 includes a pressure pad 22 having a plurality of pressure sensors 24. The pressure pad 22 is configured to be positioned between a first bone and a second bone of a joint, such as the femur ‘F’ and tibia ‘T’ of a knee joint KJ as shown in FIG. 3B. In one inventive embodiment, the pressure pad 22 is a thin film positionable between the two bones. The pressure pad 22 may be rigid, semi-rigid, and/or flexible for easy positioning. The plurality of pressure sensors 24 may illustratively include a strain gauge, capacitive sensor, electromagnetic sensor, ultrasonic sensors, piezoelectric sensor, or combinations thereof. The arrangement and density of the pressure sensors 24 may vary depending on manufacturing preferences, user preferences, and/or the particular application. In an inventive embodiment, the pressure-sensing device 20 includes a wireless transmitter 23 for transmitting data from the pressure-sensing device 20 to an external device such as a computer, display monitor, and/or a computer-assisted surgical device. In another inventive embodiment, an actuated light emitting diode (LED) 25 is used to transmit data as described in U.S. Pat. App. Publication No. US2017/0245945 assigned to the assignee of the present application. Alternatively, data from the pressure-sensing device 20 is transmitted via a wired connection. One example of a pressure-sensing device 20 for determining the pressures at various regions between two bones is the VERASENSE™ device manufactured by OrthoSensor® as described in U.S. Pat. No. 8,696,756 and incorporated by reference herein in its entirety. Further, it should be appreciated that the pressure-sensing device 20 may further provide output data in the form of a force or load.

The pressure-sensing device 20 may or may not be tracked in physical space depending on the method of performing the surgical procedure. If a computer-assisted surgical device is utilized, it may be beneficial to track the pressure-sensing device to determine the POSE of each pressure sensor 24 relative to the computer-assist device. This provides the computer-assist device with the POSE of the sensed pressure at each region between the two bones. The pressure-sensing device 20 may be tracked by a mechanical or non-mechanical tracking system. In a particular inventive embodiment, a mechanical tracking system is used. The mechanical tracking system consists of a plurality of linkages and joints to track the position of a distal link, or an end-effector attached to the distal link. Here, the pressure-sensing device 20 is assembled to the distal link to track the POSE of the pressure-sensing device 20. To accurately track the POSE of each pressure sensor 22 in space, either: a) the pressure-sensing device 20 may be assembled to the distal link in a known position and orientation; b) a calibration step may be performed to determine the relative POSEs therebetween; or c) a combination thereof. An example of a passive mechanical arm for mechanically tracking the pressure-sensing device 20 is described in U.S. Pat. No. 6,033,415 (digitizer arm 100). In a specific inventive embodiment, a non-mechanical tracking system is used to track the pressure-sensing device 20, which may include for example an optical tracking system, electromagnetic tracking system, or an acoustic tracking system. In an inventive embodiment, an optical tracking system is used as further described in more detail below. The optical tracking system detects the position of fiducial markers 26 attached or integrated with the pressure-sensing device 20. The fiducial markers may be active markers, illustratively including: light emitting diodes (LEDs) or other electromagnetic emitting markers; passive reflective markers; or a set of lines, characters, or shapes. In one inventive embodiment, the fiducial markers 26 are arranged on a rigid body to form a tracking array 28, wherein the tracking array 28 is attached to the pressure-sensing device 20. The tracking array 28 and/or fiducial markers 26 may be attached/integrated with the device 20 in a known POSE relative to the pressure sensors 22 such that the POSE of each pressure sensor can be tracked; or the relative POSE between the array/markers (28/26) may be determined with a calibration step.

For a manual TJR using cutting jigs or the like, a non-tracked pressure-sensing device 20 may be utilized to identify stability regions for stabilizing the bones during TJR. The pressure-sensing device 20 is placed between the two bones (e.g., femur ‘F’ and tibia ‘T’) as shown in FIG. 3B. At least one of the first bone or second bone is articulated throughout the bone's range of motion (ROM) where the pressures at various regions between the bones are displayed, in real time, on a display monitor. The user and/or a computer identifies at least one stability region for a given articulation angle (e.g., flexion-extension angle, abduction-adduction angle, internal-external angle). In specific inventive embodiments, a stability region is identified as a region having a higher sensed pressure compared to neighboring regions as described with reference to FIG. 3C. Therefore, the user can avoid cutting the stability regions to stabilize the first bone relative to the second bone during the TJR until all the remaining cuts have been completed around the stability region. Once the remaining cuts are completed, the bone at the stability region(s) is removed, and the user implants the implant on the modified bone.

In an embodiment of a computer-assisted TJR, the pressure-sensing device 20 and the first bone and/or second bone are tracked in space relative to a computer-assisted surgical device to aid in determining the at least one stability region. The first bone and/or second bone may be tracked with similar mechanisms as described for tracking the pressure-sensing device 20 (e.g., attaching a distal link to the first bone and/or second bone for mechanical tracking, or attaching a tracking array 28 to the first bone and/or second bone for optical tracking). The pressure-sensing device 20 is placed between the two bones, and at least one of the first bone or second bone is articulated throughout the bone's range of motion (ROM). The pressures at various regions between the bones may be recorded as well as displayed on a display monitor. The pressures at various regions between the bones may further be recorded as a function of articulation angle. With reference to FIG. 3C, an example of a series of pressure maps 30 derived from the pressure-sensing device 20 is shown. The series of pressure maps 30 includes the pressures at various regions between the bones as a function of flexion angle. For example, FIG. 3C illustrates a series of pressure maps 30 generated from pressures recorded in a knee joint ‘KJ’. On an illustrative pressure map 31, the right-to-left positions correspond to the medial-lateral positions between the femur ‘F’ and tibia ‘T’, and the up-and-down positions correspond to the anterior-posterior positions between the femur ‘F’ and tibia ‘T’. A pressure map 31 is generated for each flexion angle to generate the series of pressure maps 30. Based on the map 31, a user can identify the at least one stability region, and/or a computer may automatically identify the at least one stability region. Here, the circled regions are identified as the stability regions (32a, 32b) since the pressures are higher in these regions compared to neighboring regions. In a particular inventive embodiment, the stability regions (32a, 32b) are updated in real-time based on a current articulation angle of the bone. The cut paths are then adjusted in real-time to avoid those stability regions (32a, 32b) for the current, real-time, articulation angle. In another inventive embodiment, the pressures are averaged, at each region, over a set of articulation angles (e.g., 30°-60°) to identify the at least one stability region. A higher average for a given region over the set of articulation angles may be identified as a stability region. The cut paths are then adjusted to avoid the at least one stability region as identified from the average. An averaging method may be advantageous, as the computer does not have to keep adjusting the cut paths to avoid a stability region if the bones move during the procedure.

The at least one stability region may be identified with other methods as further described. In a particular inventive embodiment, a user may visualize the geometric relationship between the two bones to identify a stability region. For example, a user may observe the two bones and determine there is substantial contact between the two bones at the condyles. If a manual TJR procedure is conducted, the user may avoid the observed stability region. If a computer-assisted TJR procedure is conducted, the user may input the observed stability region into the computer. The user may input the observed stability region into the computer by circling or highlighting said region on virtual bone models displayed on a display monitor in the operating room. Alternatively or in combination, the user may use a digitizer to digitize the observed stability regions directly on the bones to relay the observed stability region to the computer. In another inventive embodiment, the at least one stability region may be defined pre-operatively in a planning software program. The user may identify a stability region on one or both of the virtual bone models based on their contact. In a specific inventive embodiment, finite element analysis (FEA) may be performed to determine the pressures and/or load at various regions between the two bones. The two bones may further be modeled throughout their range of motion in the planning software to determine a stability region with or without FEA analysis.

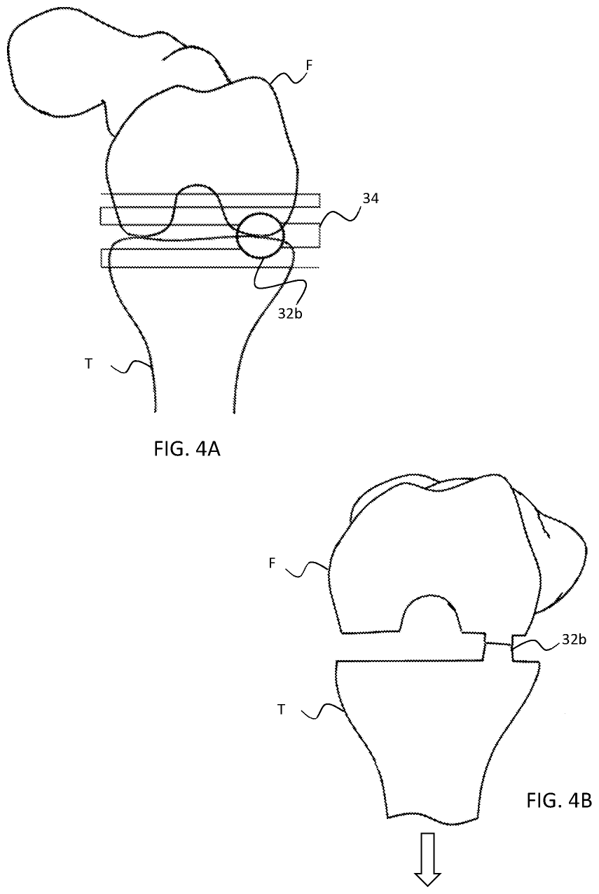

With reference to FIG. 4A, a femur ‘F’ and tibia ‘T’ are shown having a plurality of cut paths 34 positioned relative thereto and adjusted to avoid an identified stability region 32b. The plurality of cut paths 34 and the at least one stability region 32b may be determined and identified relative to the bone using any of the aforementioned methods. As shown in FIG. 4A, the cut paths 34 skip over the circled stability region 32b. With reference to FIG. 4B, the femur ‘F’ and tibia ‘T’ are shown having all the remaining cut paths completed leaving the stability region 32b intact. Therefore, the femur ‘F’ is stabilized against the tibia ‘T’ as the remaining cut paths 34 are executed around the stability region 32b. In other words, the two bones do not collapse towards one another because of the contact therebetween at the stability region 32b. Otherwise, the bones would collapse and be forced towards one another by the ligaments and surrounding tissues, which would inhibit or at least make bone removal/cutting therebetween difficult.

In a specific inventive embodiment, for a manual TJR, the user may avoid cutting the at least one stability region 32b while executing all the remaining cuts. Once removed, the final stability region 32b is removed to implant the implant thereon.

In a particular inventive embodiment of a computer-assisted TJR, a computer may adjust the cut paths to avoid the at least one stability region 32b. More specifically, for an autonomous/active robot assisted TJR with the surgical system as described below, a computing system (e.g., controller) of the robot may execute a collision avoidance algorithm to avoid the identified stability region 32b. The robot may be programmed to executed the plurality of cut paths 34 on the bones autonomously, where the at least one stability region 32b may be modeled as an obstacle in the cut path, such that the collision avoidance algorithm causes the end-effector to least one of: (a) stop prior to colliding with the stability region 32b; and/or (b) re-route around the stability region 32b. If re-routed, the end-effector may be re-routed around the perimeter of the stability region 32b. In other embodiments, the robot may be programmed with a plurality of different cut-files, each cut-file having a different set of cut paths. Once at least one stability region is identified 32b, the computer determines which cut-file contains the correct cut paths to create the bone cuts and avoid the stability region 32b.

In another embodiment of a computer-assisted TJR, a computer may haptically constrain an end-effector of a robotic arm from entering into the stability region 32b. The end-effector may be manually wielded by a user but constrained within virtual boundaries by providing haptic forces against the user when the user tries to cross the virtual boundary. A virtual boundary may therefore be defined as the perimeter of the stability region 32b constraining the user to cut paths around the stability region 32b. One such haptic computer-assisted surgical device is the RIO® Robotic Arm manufactured by Stryker/MAKO and described in U.S. Pat. No. 7,689,014.

In a further embodiment of a computer-assisted TJR, a computer may provide power control over a surgical device. The surgical device may include a tracked burr or other cutting instrument operated by a drill. As the cutting device encounters the perimeter or boundary of the stability region 32b, the power to the drill is terminated. Therefore the cutting device is unable to cut the bone in the stability region 32b. The cutting device may further be retractable within a drill guard and/or a drill guard may be extendable over the cutting device. If the cutting device encounters the perimeter or boundary of the stability region, then the cutting device is retracted into the drill guard and/or the drill guard is extended over the cutting device. One such computer-assisted drill device is the NavioPFS® System manufactured by Smith & Nephew and described in U.S. Pat. No. 6,757,582.

After the remaining cut paths have been executed around the at least one stability region as shown in FIG. 4B, the stability region 32b may be removed. The stability region 32b may be removed using manual tools such as a rongeur, saw, drill, or the like. In other inventive embodiments, the stability region 32b may be removed with a computer-assisted surgical device as described above and below. Once the stability region 32b is removed, the user may implant the implant on the bone in the desired/planned position and orientation. In some inventive embodiments, at least one of the bones defining the stability region 32b is placed in traction during the removal. In still other embodiments, the traction is directed away from the cut plane, as depicted by the arrow in FIG. 4B. Traction is noted to inhibit dynamic changes in the cutting region as the stability region 32b is removed.

### Bone Stability Using a Wedge

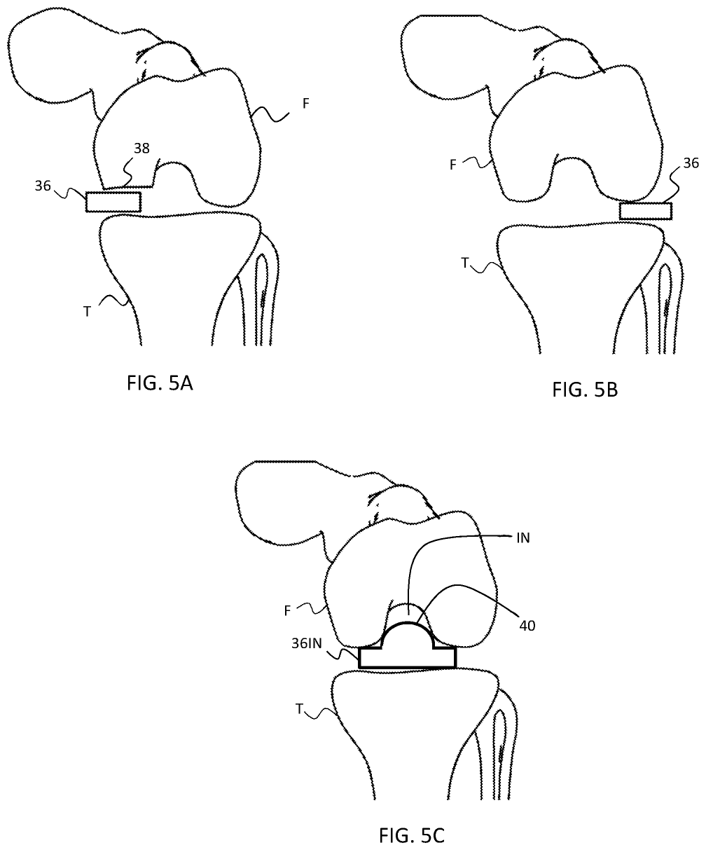

In specific inventive embodiments, with reference to FIGS. 5A and 5B, the bones may be stabilized using a wedge 36. FIG. 5A depicts a wedge 36 inserted between the tibia T and a cut surface 38 on the femur F. FIG. 5B depicts a wedge 36 inserted between a native femur F and tibia T. The wedge 36 may be a retractor, a block, a wedged instrument, or other device capable of being inserted between two bones of a joint. The wedge 36 stabilizes the position of a first bone relative to a second bone during TJR to ensure either bone does not interfere or collapse on a cutting device while cutting the bone.

With reference to FIG. 5C a wedge 36IN may be positioned in the region of intercondylar notch IN of the femur F. The wedge 36IN has a conical section 40 that self-locates in the intercondylar notch IN of the femur F. The use of the intercondylar notch IN of the femur F for locating a wedge is advantageous in that this area is typically not shaped much by the cutting blade, and by shimming in the middle, the gap is better maintained by the wedge and does not need to be moved as often during surgery. Furthermore, the tibial surface is also kept mostly clear for cutting compared to the placement of wedges 36 as shown in FIGS. 5A and 5B.

A method of using the wedge 36 may include the following steps. Operational data (e.g., a set of instructions for a robot arm, a set of virtual boundaries to constrain a robot arm, a set of planes or lines in which an end-effector is to be maintained) is generated for a computer-assisted surgical device to remove bone according to a surgical plan. The operational data may be partitioned to remove one or more segments of bone at one time. At least a portion of the bone is removed to create at least one cut surface 38 on the bone. A wedge 36 is inserted between the at least one cut surface 38 and the opposing bone to stabilize the bones relative to one another. The remaining bone is then removed with the wedge 36 inserted to complete the preparation of the bone to receive an implant.

The operational data may be partitioned based one or more stability regions of the bone. One or more stability regions may be identified pre-operatively or intra-operatively as described above, where the operational data is partitioned to remove bone at the stability region first. The computer-assisted surgical device then cuts the stability region first, and the wedge 36 is inserted at the stability region before removing the remaining bone. Thus the wedge 36 is inserted at an area of good bone quality where the bones can be supported.

Another method of using the wedge 36 may include the following steps. A wedge 36 is inserted between two uncut bones of a joint as shown in FIG. 5B. A computer-assisted surgical device then removes bone around the wedge 36. The wedge 36 is removed and the remaining bone is removed where the wedge 36 was inserted.

Several techniques may be utilized to ensure the computer-assisted surgical device does not collide with the wedge 36 when inserted between uncut bone. In a particular embodiment, the location for the wedge 36 between the bones may be pre-operatively planned. The pre-operative planning software may include a virtual model or outline of the wedge 36 to be virtually positioned between the bones to designate the location for the wedge 36. The operational data is then generated and partitioned to avoid the pre-planned location for the wedge 36 until all of the other bone has been removed therearound. The user may further plan the location for the wedge 36 based on one or more stability regions. In another embodiment, the wedge 36 may be tracked in physical space by a tracking system (e.g., optical tracking system). A tracking array may be attached/integrated with the wedge 36 such that the surgical system knows the POSE of the wedge 36 in real-time. The surgical device may be equipped with a control system and/or collision avoidance software that avoid the POSE of the wedge 36 in real-time. In a further embodiment, the computer-assisted surgical device may be held and wielded by the user while the surgical device provides haptic control, power control, or semi-active control (e.g., a 1-N degree-of-freedom hand-held surgical device). In this scenario, the user may simply avoid the wedge 36 while removing bone therearound.

### Surgical System

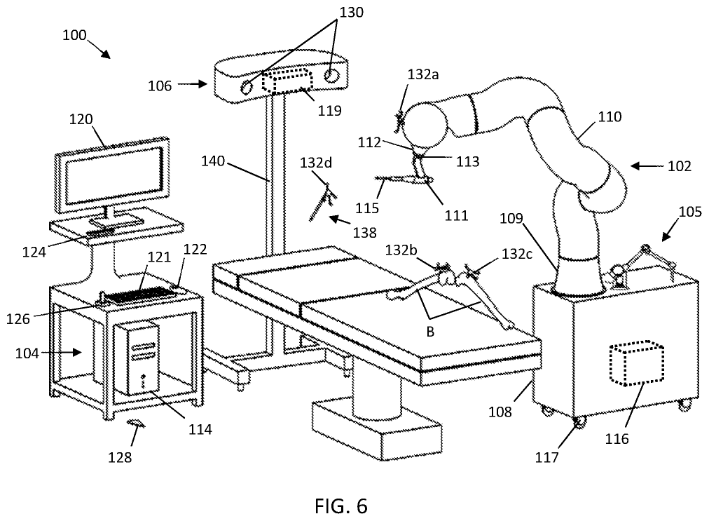

With reference to FIG. 6, an example of a computer-assisted surgical system 100 in the context of an operating room (OR) is shown. The surgical system 100 generally includes a surgical robot 102, a computing system 104, a mechanical arm 105 and/or a non-mechanical tracking system 106 (e.g., an optical tracking system, an electro-magnetic tracking system), and a rollable or handheld digitizer 138.

The surgical robot 102 may include a movable base 108, a manipulator arm 110 connected to the base 108, an end-effector flange 112 located at a distal end of the manipulator arm 110, and an end-effector assembly 111 removably attached to the flange 112 by way of an end-effector mount/coupler 113. The end-effector assembly 111 holds and/or operates an end-effector tool 115 that interacts with a portion of a patient's anatomy. The base 108 includes a set of wheels 117 to maneuver the base 108, which may be fixed into position using a braking mechanism such as a hydraulic brake. The base 108 may further include an actuator 109 to adjust the height of the manipulator arm 110. The manipulator arm 110 includes various joints and links to manipulate the tool 115 in various degrees of freedom. The joints are illustratively prismatic, revolute, spherical, or a combination thereof.

The computing system 104 generally includes a planning computer 114; a device computer 116; an optional tracking computer 119 if a tracking system 106 is present; and peripheral devices. The planning computer 114, device computer 116, and tracking computer 119, may be separate entities, single units, or combinations thereof depending on the surgical system. The peripheral devices allow a user to interface with the robotic surgical system 100 and may include: one or more user-interfaces, such as a display or monitor 120; and user-input mechanisms, such as a keyboard 121, mouse 122, pendent 124, joystick 126, foot pedal 128, or the monitor 120 in some inventive embodiments may have touchscreen capabilities.

The planning computer 114 contains hardware (e.g., processors, controllers, and memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, modeling the bones range-of-motion, executing finite element analysis, determining stability regions, and generating surgical plan data. The final surgical plan includes operational data for modifying a volume of tissue that is defined relative to the anatomy, illustratively including: a set of points in a cut-file to autonomously modify the volume of bone; a set of virtual boundaries defined to haptically constrain a tool within the defined boundaries to modify the bone; a set of planes or drill holes to drill pins in the bone; or a graphically navigated set of instructions for modifying the tissue. In particular inventive embodiments, the operational data specifically includes a cut-file for execution by a surgical robot to autonomously or automatically modify the volume of bone, which is advantageous from an accuracy and usability perspective. The data generated from the planning computer 114 may be transferred to the device computer 116 and/or tracking computer 119 through a wired or wireless connection in the operating room (OR); or transferred via a non-transient data storage medium (e.g., a compact disc (CD), a portable universal serial bus (USB) drive).

The device computer 116 in some inventive embodiments is housed in the moveable base 108 and contains hardware (e.g., controllers), software, data and utilities that are preferably dedicated to the operation of the surgical robot 102. This may include surgical device control, robotic manipulator control, the processing of kinematic and inverse kinematic data, the execution of registration algorithms, the execution of calibration routines, the execution of surgical plan data, the execution of operational data, coordinate transformation processing, providing workflow instructions to a user, utilizing position and orientation (POSE) data from the tracking system 106, and reading data received from the mechanical arm 105.

The optional tracking system 106 of the surgical system 100 may be an optical tracking system as described in U.S. Pat. No. 6,061,644. The optical tracking system includes two or more optical receivers 130 to detect the position of tracking arrays (28, 132a, 132b, 132c), where each tracking array (28, 132a, 132b, 132c) has a unique arrangement of fiducial markers 26, or a unique transmitting wavelength/frequency if the markers 26 are active LEDs. The tracking system 106 may be built into a surgical light, located on a boom, a stand 140, or built into the walls or ceilings of the OR. The tracking system computer 119 may include tracking hardware, software, data and utilities to determine the POSE of objects (e.g., bones B, rollable or handheld digitizer 138, and surgical robot 102) in a local or global coordinate frame. The POSE of the objects is collectively referred to herein as POSE data, where this POSE data may be communicated to the device computer 116 through a wired or wireless connection. Alternatively, the device computer 116 may determine the POSE data using the position of the fiducial markers detected from the optical receivers 130 directly.

The POSE data is used by the computing system 104 during the procedure to update the POSE and/or coordinate transforms of the bone B, the surgical plan, and the surgical robot 102 as the manipulator arm 110 and/or bone B move during the procedure, such that the surgical robot 102 can accurately execute the surgical plan. In another embodiment, the surgical system 100 does not include a tracking system 106, but instead employs a mechanical arm 105, and a bone fixation and monitoring system that fixes the bone directly to the surgical robot 102 and monitors bone movement as described in U.S. Pat. No. 5,086,401.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method of stabilizing a first bone relative to a second bone in a joint during total joint arthroplasty, comprising:
determining a plurality of cut paths relative to the first bone in order to modify the first bone to receive an implant thereon;
identifying one or more stability regions between the first bone and the second bone; and
adjusting a projected one or more cut paths to avoid at least one of the one or more stability regions, wherein the first bone is stabilized against the second bone at the at least one of the one or more stability regions while the remaining cut paths are executed around the at least one of the one or more stability regions.

2. The method of claim 1 wherein identifying the one or more stability regions comprises:
inserting a pressure-sensing device between the first bone and the second bone;
articulating at least one of the first bone or the second bone throughout a range of motion (ROM);
recording pressures at various regions between the first bone and the second bone throughout the ROM; and
identifying the one or more stability regions based on the recorded pressures.

3. The method of claim 2 wherein the pressure-sensing device includes a plurality of pressure sensors, said pressure sensors being at least one of a strain gauge, capacitive sensor, electromagnetic sensor, or piezoelectric sensor.

4. The method of claim 2 further comprising:
tracking the pressure-sensing device relative to the first bone and second bone by a computer-assisted device; and
generating a series of pressure maps having the pressures at various regions between the first bone and the second bone, wherein said pressures at each region is known relative to the computer-assisted surgical device.

5. The method of claim 4 wherein the computer-assisted device comprises an optical tracking system.

6. The method of claim 1 wherein identifying the one or more stability regions comprises:
generating a virtual model of the first bone and the second bone;
performing finite element analysis to determine the pressures at various regions between the first bone and the second bone; and
identifying the one or more stability regions based on the finite element analysis.

7. The method of claim 1 wherein the one or more stability regions are identified as a function of articulation angle, wherein a first articulation angle includes a first set of one or more stability regions and a second articulation angle includes a second set of one or more stability regions.

8. The method of claim 1 wherein the determination of the plurality of cut paths comprises:
generating a first bone model of the first bone and a second bone model of the second bone; and
planning a desired position for an implant model relative to at least one of the first bone model and second bone model, wherein the plurality of cutting paths is determined relative to the first bone based on the plan.

9. The method of claim 1 wherein the plurality of cut paths are executed by a robotic surgical system.

10. The method of claim 1 further comprising transferring the determined cut paths to a robotic surgical system, said robotic surgical system comprising a manipulator arm supporting an end-effector, a computing system for controlling the manipulator arm along the cut paths, and a tracking system.

11. The method of claim 1 further comprising removing the at least one of the one or more stability regions once the remaining cut paths are completed.

12. The method of claim 1 further comprising executing the remaining cutting paths around the at least one stability region with the robotic surgical system.

13. The method of claim 1 wherein the execution of the remaining cutting paths is performed by the robotic surgical system autonomously.

14. The method of claim 1 wherein the removing of the at least one of the one or more stability regions occurs while one of the first bone or the second bone is under traction.

15. The method of claim 14 wherein the traction is elongating relative to the one or more stability regions.

16. A system for performing the method of claim 1, comprising:
a pressure-sensing device to aid in identifying the at least one stability region;
a manipulator arm supporting an end-effector;
a computing system comprising a plurality of cut files stored therein, each cut file having a set of cut paths to be executed by the manipulator arm; and
wherein said computing system selects a specific cut file based on the output from the pressure-sensing device, said specific cut file having a set of cut paths that avoid the at least one stability region.

17. A computer-assisted surgical system, comprising:
a computer-assisted surgical device having an end-effector;
a computing system comprising operational data to be executed by the surgical device to remove bone according to a surgical plan; and
a wedge to be inserted between two bones to stabilize the bones while the surgical device removes bone according to a method of claim 11.

18. The computer-assisted surgical system of claim 17 wherein the computer-assisted surgical device is a robot manipulator arm and the operational data is a plurality of cut files, each cut file having a set of cut paths to be executed by the manipulator arm; and
wherein said computing system selects a specific cut file based on a position of the wedge between the bones that avoids the wedge.

19. The computer-assisted surgical system of claim 18 wherein the wedge is at least one of a retractor, a block, or a wedged instrument.

20. The computer-assisted surgical system of claim 17 wherein the wedge further comprises a conical section that self-locates in a patient's intercondylar notch of the patient's femur.
